(19) United States
(12) Patent Application Publication
(10) Pub. No.: US 2007/0185339 A1
(43) Pub. Date: Aug. 9, 2007

(54) PROCESS FOR TREATING A CATALYST, THE CATALYST, AND USE OF THE CATALYST

(76) Inventor: Jian LU, Houston, TX (US)

Correspondence Address: SHELL OIL COMPANY, P O BOX 2463, HOUSTON, TX, 772522463, US

(21) Appl. No.: 11/670,325

(22) Filed: Feb. 1, 2007

Related U.S. Application Data
(60) Provisional application No. 60/764,992, filed on Feb. 3, 2006.

(51) Int. Cl. C07D 301/10; B01J 38/04

(52) U.S. Cl. 549/534; 502/34

(57) ABSTRACT

A process for treating a supported epoxidation catalyst comprising silver in a quantity of at most 0.15 g per m² surface area of the support, which process comprises:
contacting the catalyst, or a precursor of the catalyst comprising silver in cationic form, with a treatment feed comprising oxygen at a catalyst temperature of at least 350° C. for a duration of at least 5 minutes;
   the catalyst;
   a process for the epoxidation of an olefin; and
   a process for producing a 1,2-diol, 1,2-diol ether, or an alkanolamine.

### CROSS-REFERENCE TO RELATED APPLICATIONS

This application claims the benefit of U.S. Provisional Application No. 60/764,992, filed Feb. 3, 2006, the entire disclosure of which is hereby incorporated by reference.

### FIELD OF THE INVENTION

The invention relates to a process for treating a catalyst, the catalyst, and a process for the production of an olefin oxide, a 1,2-diol, a 1,2-diol ether, or an alkanolamine.

### BACKGROUND OF THE INVENTION

In olefin epoxidation, an olefin is reacted with oxygen in the presence of a silver-based catalyst to form the olefin epoxide. The olefin oxide may be reacted with water, an alcohol or an amine to form a 1,2-diol, a 1,2-diol ether or an alkanolamine. Thus, 1,2-diols, 1,2-diol ethers and alkanolamines may be produced in a multi-step process comprising olefin epoxidation and converting the formed olefin oxide with water, an alcohol or an amine.

Modern silver-based catalysts are more highly selective towards olefin oxide production. When using the modern catalysts in the epoxidation of ethylene, the selectivity towards ethylene oxide can reach values above the 6/7 or 85.7 mole-% limit. This limit has long been considered to be the theoretically maximal selectivity of this reaction, based on the stoichiometry of the reaction equation

7C₂H₄+6O₂=>6C₂H₄O+2CO₂+2H₂O,

cf. Kirk-Othmer's Encyclopedia of Chemical Technology, 3^{rd} ed., Vol. 9, 1980, p. 445. Such highly selective catalysts may comprise as their active components silver, and one or more selectivity enhancing dopants, such as components comprising rhenium, tungsten, chromium or molybdenum. Highly selective catalysts are disclosed, for example, in U.S. Pat. No. 4,761,394 and U.S. Pat. No. 4,766,105.

During the initial phase of an epoxidation process, the catalyst experiences the so-called “break-through phase” during which the oxygen conversion is very high, and the level of selectivity is very low, even in the presence of a reaction modifier. The epoxidation process is difficult to control during this break-through phase. In particular, it may take a long time in the initial phase of a commercial epoxidation process for the conversion to drop so that the reaction can more easily be controlled at an attractive level of the selectivity.

U.S. Patent Application 2004/0049061 discusses improving selectivity of a highly selective silver-based catalyst, containing at most 0.17 g/m² surface area, by heating the catalyst above 250° C. for up to 150 hours in the presence of oxygen. The temperatures disclosed in a preferred embodiment are in the range of from above 250° C. to at most 320° C.

U.S. Patent Application 2004/0110971 relates to improving the start-up of an epoxidation process, i.e., reducing the duration of the break-through phase occurring during the initial phase of the epoxidation process, by contacting the highly selective catalyst with an oxygen feed at a temperature above 260° C. for a period of at most 150 hours. The temperatures disclosed in a preferred embodiment are in the range of from above 260° C. to at most 300° C.

Thus, a desire exists for process improvements which further improve selectivity and reduce the duration of the break-through phase occurring during the initial phase of the epoxidation process.

During the start-up of a commercial epoxidation process, additional procedures may be employed. For example, it may be useful to pre-treat catalysts prior to carrying out the epoxidation process by subjecting them to a high temperature, i.e., in the range of from 200 to 250° C., with an inert sweeping gas passing over the catalyst. The sweeping gas comprises nitrogen, argon and mixtures thereof. The high catalyst temperature converts a significant portion of organic nitrogen compounds which may have been used in the manufacture of the catalysts to nitrogen containing gases which are swept up in the gas stream and removed from the catalyst.

Additionally, it may be useful during the start-up of a commercial epoxidation process to pre-soak the catalyst with a feed comprising a reaction modifier, in particular an organic halide, and then contact the catalyst with a feed comprising a reaction modifier at a low concentration, if any.

A desire also exists for more efficient start-up processes which do not require such pre-treat and/or pre-soak procedures.

Another important characteristic of an epoxidation catalyst is the mechanical strength of the catalyst since catalysts with low mechanical strength can cause problems within the commercial processes. Mechanical strength can include attrition resistance and crush strength.

Within commercial processes, friction or rubbing occurs between the catalyst particles themselves or between the catalyst and equipment surfaces. This friction or rubbing may occur during catalyst manufacturing, catalyst shipping, epoxidation reactor loading, or other reactor processes. These forces can cause the catalyst to breakdown into smaller particles called fines. This physical breakdown of the catalyst is known as attrition.

Attrition occurring during the loading of the catalyst into the epoxidation reactor may cause dusting problems which results in a loss of valuable catalyst. The difficulty associated with attrition with respect to the epoxidation process is that the fines may be driven away from the reaction zone, resulting in 1) excessive developments of the reaction in the separators or other locations within the oxidation process and 2) creating problems in the recovery systems. The loss of catalyst reduces the productivity of the catalyst bed effecting overall process efficiency and increasing operating costs. Thus, it would be highly desirable to improve the attrition resistance of catalysts.

It is also desirable to improve the crush strength of the catalyst. Within commercial processes, large forces are exerted on the catalyst during the loading of the reactor and during the course of the reaction. Breakage of the catalysts in the reactor leads to increased pressure drop and poor distribution of the reactants over the catalyst bed.

EP-A-808215 teaches that catalysts prepared with a carrier made by utilizing polypropylene as a burnout material have improved crush strength and attrition resistance.

U.S. Pat. No. 4,428,863 teaches incorporating barium aluminate and barium silicate into the carrier to improve crush strength and attrition resistance.

Thus, notwithstanding the improvements already achieved, there is a desire to improve the performance of olefin epoxidation catalysts and, in particular, to increase the mechanical strength of the catalysts.

### SUMMARY OF THE INVENTION

The invention provides a process for treating a supported epoxidation catalyst comprising silver in a quantity of at most 0.15 g per m² surface area of the support, which process comprises:
contacting the catalyst, or a precursor of the catalyst comprising silver in cationic form, with a treatment feed comprising oxygen at a catalyst temperature of at least 350° C. for a duration of at least 5 minutes.

The invention also provides an epoxidation catalyst which is obtainable by the process in accordance with this invention.

The invention also provides a process for the epoxidation of an olefin, which process comprises contacting an epoxidation feed comprising the olefin and oxygen with an epoxidation catalyst prepared in accordance with this invention.

The invention also provides a process for producing a 1,2-diol, a 1,2-diol ether or an alkanolamine comprising converting the olefin oxide into the 1,2-diol, the 1,2-diol ether, or the alkanolamine, wherein the olefin oxide has been obtained by a process for the epoxidation of an olefin in accordance with this invention.

### BRIEF DESCRIPTION OF THE FIGURE

FIG. 1 shows the selectivity (“S (%)”) as a function of time, in days, (“T, (D)”), as observed in Example 1, Example 2 and Example 3 (referenced as “1”, “2” and “3” respectively).

### DETAILED DESCRIPTION OF THE INVENTION

Catalysts treated by a process in accordance with this invention can exhibit improved mechanical strength, as may be found by attrition and/or crush strength tests.

Additionally, catalysts treated by a process in accordance with this invention and which further comprise one or more selectivity enhancing dopants, exhibit improved catalytic performance, in particular increased initial selectivity. Also, these treated catalysts, which further comprise one or more selectivity enhancing dopants, can exhibit an initial selectivity at an earlier stage in the epoxidation process which results in additional production of olefin oxide.

As an additional advantage, the procedure of pre-treating the catalyst with a sweeping gas may be eliminated during the start-up of the epoxidation process. Also, the procedure of pre-soaking the catalyst with a reaction modifier may become unnecessary and may, therefore, be eliminated. These additional advantages improve process efficiency and lower operating costs.

As used herein, initial selectivity is meant to be the maximum selectivity achieved after the catalyst has been placed on stream. In the practice of using catalysts in accordance with this invention, the initial selectivity is reached before about 72 hours of operation. As exemplified herein, the initial selectivity may be measured at an olefin oxide make of 1.7% at the reactor outlet and at a gas hourly space velocity of approximately 6800 Nl/(l.h).

The support material for use in this invention may be natural or artificial inorganic particulate materials and they may include refractory materials, silicon carbide, clays, zeolites, charcoal and alkaline earth metal carbonates, for example calcium carbonate or magnesium carbonate. Preferred are refractory materials, such as alumina, magnesia, zirconia and silica. The most preferred material is α-alumina. Typically, the support material comprises at least 85% w, more typically 90% w, in particular 95% w α-alumina or a precursor thereof, frequently up to 99.9% w, or even up to 100% w, α-alumina or a precursor thereof. The α-alumina may be obtained by mineralization of α-alumina, suitably by boron or, preferably, fluoride mineralization. Reference is made to U.S. Pat. No. 3,950,507, U.S. Pat. No. 4,379,134 and U.S. Pat. No. 4,994,589, which are incorporated herein by reference.

Precursors of support materials may be chosen from a wide range. For example, α-alumina precursors include hydrated aluminas, such as boehmite, pseudoboehmite, and gibbsite, as well as transition aluminas, such as the chi, kappa, gamma, delta, theta, and eta aluminas.

The support material may preferably have a surface area of at most 20 m²/g, in particular in the range of from 0.5 to 20 m²/g, more in particular from 1 to 10 m²/g, and most in particular from 1.5 to 5 m²/g. “Surface area” as used herein is understood to refer to the surface area as determined by the BET (Brunauer, Emmett and Teller) method as described in Journal of the American Chemical Society 60 (1938) pp. 309-316.

In an embodiment, the alumina support has a surface area of at least 1 m²/g, and a pore size distribution such that pores with diameters in the range of from 0.2 to 10 μm represent at least 70% of the total pore volume and such pores together provide a pore volume of at least 0.25 ml/g, relative to the weight of the support. Preferably in this particular embodiment, the pore size distribution is such that pores with diameters less than 0.2 μm represent from 0.1 to 10% of the total pore volume, in particular from 0.5 to 7% of the total pore volume; the pores with diameters in the range of from 0.2 to 10 μm represent from 80 to 99.9% of the total pore volume, in particular from 85 to 99% of the total pore volume; and the pores with diameters greater than 10 μm represent from 0.1 to 20% of the total pore volume, in particular from 0.5 to 10% of the total pore volume. Preferably in this particular embodiment, the pores with diameters in the range of from 0.2 to 10 μm provide a pore volume in the range of from 0.3 to 0.8 ml/g, in particular from 0.35 to 0.7 ml/g. Preferably in this particular embodiment, the total pore volume is in the range of from 0.3 to 0.8 ml/g, in particular from 0.35 to 0.7 ml/g. Preferably in this particular embodiment, the surface area of the support is at most 3 m²/g. Preferably in this particular embodiment, the surface area is in the range of from 1.4 to 2.6 m²/g.

In another embodiment, the alumina support has a surface area of at least 1 m²/g, and a pore size distribution such that the median pore diameter is more than 0.8 μm, and such that at least 80% of the total pore volume is contained in pores with diameters in the range of from 0.1 to 10 μm, and at least 80% of the pore volume contained in the pores with diameters in the range of from 0.1 to 10 μm is contained in pores with diameters in the range of from 0.3 to 10 μm. Preferably in this particular embodiment, the pore size distribution is such that pores with diameters less than 0.1 μm represent at most 5% of the total pore volume, in particular at most 1% of the total pore volume; the pores with diameters in the range of from 0.1 to 10 μm represent less than 99% of the total pore volume, in particular less than 98% of the total pore volume; the pores with diameters in the range of from 0.3 to 10 μm represent at least 85%, in particular at least 90% of the pore volume contained in the pores with diameters in the range of from 0.1 to 10 μm; the pores with diameters less than 0.3 μm represent from 0.01 to 10% of the total pore volume, in particular from 0.1 to 5% of the total pore volume; and the pores with diameters greater than 10 μm represent from 0.1 to 10% of the total pore volume, in particular from 0.5 to 8% of the total pore volume. Preferably in this particular embodiment, the pore size distribution is such that the median pore diameter is in the range of from 0.85 to 1.9 μm, in particular 0.9 to 1.8 μm. Preferably in this particular embodiment, the surface area of the support is at most 3 m²/g. Preferably in this particular embodiment, the surface area is in the range of from 1.4 to 2.5 m²/g.

As used herein, the pore size distribution and the pore volumes are as measured by mercury intrusion to a pressure of 3.0×10⁸ Pa using a Micromeretics Autopore 9200 model (130° contact angle, mercury with a surface tension of 0.473 N/m, and correction for mercury compression applied).

As used herein, the median pore diameter is the pore diameter at which half of the total pore volume is contained in pores having a larger pore diameter and half of the total pore volume is contained in pores having a smaller pore diameter.

As used herein, pore volume (ml/g), and surface area (m²/g) and water absorption (g/g) are defined relative to the weight of the carrier, unless stated otherwise.

In an embodiment, the support material or precursor thereof may have been treated, in particular in order to reduce its ability to release sodium ions, i.e. to reduce its sodium solubilization rate, or to decrease its content of water soluble silicates. A suitable treatment comprises washing with water. For example, the support material or precursor thereof may be washed in a continuous or batch fashion with hot, demineralised water, for example, until the electrical conductivity of the effluent water does not further decrease, or until in the effluent the content of sodium or silicate has become very low. A suitable temperature of the demineralised water may be in the range of 80 to 100° C., for example 90° C. or 95° C. Alternatively, the support material or precursor thereof may be washed with base and subsequently with water. After washing, the support material or precursor thereof may typically be dried. Reference may be made to U.S. Pat. No. 6,368,998, which is incorporated herein by reference. Catalysts which have been prepared by using the support material or precursor material that has been so treated have an improved performance in terms of an improved initial selectivity, initial activity and/or stability, in particular selectivity stability and/or activity stability.

The attrition test as referred to herein is in accordance with ASTM D4058-96, wherein the test sample is tested as such after its preparation, that is with elimination of Step 6.4 of the said method, which represents a step of drying the test sample. The attrition loss measured for the catalyst prepared in accordance to the invention may preferably be at most 50%, more preferably at most 40%, most preferably at most 30%, in particular at most 20%. Frequently, the attrition loss may be at least 10%.

The crush strength as referred herein is as measured in accordance with ASTM D6175-98, wherein the test sample is tested as such after its preparation, that is with elimination of Step 7.2 of the said method, which represents a step of drying the test sample. The crush strength of the catalyst prepared in accordance with the invention, in particular when measured as the crush strength of hollow cylindrical particles of 8.8 mm external diameter and 3.5 mm internal diameter, may be at least 2 N/mm, preferably at least 4 N/mm, more preferably at least 6 N/mm, and most preferably at least 8 N/mm. The crush strength, in particular when measured as the crush strength of hollow cylindrical particles of 8.8 mm external diameter and 3.5 mm internal diameter, may be frequently at most 25 N/mm, in particular at most 20 N/mm. The catalyst particles having the shape of the particular hollow cylinder have a cylindrical bore, defined by the internal diameter, which is co-axial with the external cylinder. Such catalyst particles, when they have a length of about 8 mm, are frequently referred to as “nominal 8 mm cylinders”, or “standard 8 mm cylinders”.

Generally, it is found very convenient to use catalyst particles, for example, in the form of trapezoidal bodies, cylinders, saddles, spheres, doughnuts. The catalyst particles may typically have a largest outer dimension in the range of from 3 to 15 mm, preferably from 5 to 10 mm. They may be solid or hollow, that is they may have a bore. Cylinders may be solid or hollow, and they may have a length typically from 3 to 15 mm, more typically from 5 to 10 mm, and they may have a cross-sectional, outer diameter typically from 3 to 15 mm, more typically from 5 to 10 mm. The ratio of the length to the cross-sectional diameter of the cylinders may typically be in the range of from 0.5 to 2, more typically from 0.8 to 1.25. The shaped particles, in particular the cylinders, may be hollow, having a bore typically having a diameter in the range of from 0.1 to 5 mm, preferably from 0.2 to 2 mm. The presence of a relatively small bore in the shaped particles increases their crush strength and the achievable packing density, relative to the situation where the particles have a relatively large bore. The presence of a relatively small bore in the shaped particles is beneficial in the drying of the shaped catalyst, relative to the situation where the particles are solid particles, that is having no bore.

Preferably, the catalysts comprise, in addition to silver, a Group IA metal, and one or more selectivity enhancing dopants which may be selected from rhenium, molybdenum and tungsten. The catalysts which comprise a selectivity enhancing dopant are designated herein as “highly selective catalysts.”

The catalysts comprise silver suitably in a quantity of from 10 to 500 g/kg, more suitably from 50 to 300 g/kg, on the total catalyst. The Group IA metals, as well as the selectivity enhancing dopants, may each be present in a quantity of from 0.01 to 500 mmole/kg, calculated as the element (rhenium, molybdenum, tungsten or Group IA metal) on the total catalyst. Preferably, the Group IA metal may be selected from lithium, potassium, rubidium and cesium. Rhenium, molybdenum or tungsten may suitably be provided as an oxyanion, for example, as a perrhenate, molybdate, tungstate, in salt or acid form.

Preferably, the quantity of silver relative to the surface area of the support, i.e., silver density, may be at most 0.15 g/m², more preferably at most 0.14 g/m², most preferably at most 0.12 g/m², for example at most 0.1 g/m². Preferably, the quantity of silver relative to the surface area of the support may be at least 0.01 g/m², more preferably at least 0.02 g/m². Without wishing to be bound by theory, the catalysts having a low silver density on the support surface may exhibit minimum contact sintering during the heat treatment of the catalysts.

Of special preference are the highly selective epoxidation catalysts which comprise rhenium, in addition to silver. The highly selective epoxidation catalysts are known from U.S. Pat. No. 4,761,394 and U.S. Pat. No. 4,766,105, which are incorporated herein by reference. Broadly, they comprise silver, rhenium or compound thereof, a further metal or compound thereof and optionally a rhenium co-promoter which may be selected from one or more of sulfur, phosphorus, boron, and compounds thereof, on the support material. The further metal may be selected from Group IA metals, Group IIA metals, molybdenum, tungsten, chromium, titanium, hafnium, zirconium, vanadium, thallium, thorium, tantalum, niobium, gallium, germanium, and mixtures thereof. Preferably the Group IA metals may be selected from lithium, potassium, rubidium, and cesium. The Group IIA metals may be selected from calcium and barium. Most preferably the Group IA metals may be selected from lithium, potassium and/or cesium. Where possible, rhenium, the further metal or the rhenium co-promoter may typically be provided as an oxyanion, in salt or acid form.

Preferred amounts of the components of these catalysts are, when calculated as the element on the total catalyst:

silver from 10 to 500 g/kg,

rhenium from 0.01 to 50 mmole/kg,

the further metal or metals from 0.1 to 500 mmole/kg each, and, if present,

the rhenium co-promoter or co-promoters from 0.1 to 30 mmole/kg each.

The preparation of the catalysts is known in the art and the known methods are applicable to this invention. Methods of preparing the catalyst include impregnating the support with a silver compound and with other catalyst ingredients, and performing a reduction to form metallic silver particles. Reference may be made, for example, to U.S. Pat. No. 4,761,394, U.S. Pat. No. 4,766,105, U.S. Pat. No. 5,380,697, U.S. Pat. No. 5,739,075, U.S. Pat. No. 6,368,998, US-2002/0010094 A1, WO-00/15333, WO-00/15334 and WO-00/15335, which are incorporated herein by reference.

The heat treatment of this invention may be applied to a catalyst or to a precursor of the catalyst. By a precursor of the catalyst is meant the supported composition which comprises the silver in unreduced, i.e. cationic form, and which further comprises the components necessary for obtaining the intended catalyst after reduction. In this case, the reduction may be effected during the contacting with a treatment feed, as discussed herein.

The heat treatment of this invention may also be applied to catalysts during their use in an epoxidation process, or to used catalysts which, due to a plant shut-down, have been subjected to a prolonged shut-in period; however, most commercial plants do not contain systems capable of heating the catalyst to the temperatures required by the present invention.

As used herein, the catalyst temperature is deemed to be the weight average temperature of the catalyst particles.

In accordance with this invention, the catalyst, or a precursor of the catalyst comprising silver in cationic form, is treated by contacting it with a treatment feed comprising oxygen at a catalyst temperature of at least 350° C., which treatment may herein be referred to by the term “heat treatment”. Preferably, a catalyst temperature above 350° C., more preferably at least 375° C., most preferably at least 400° C. may be employed. Preferably, a catalyst temperature of at most 700° C., more preferably at most 600° C., most preferably at most 500° C., may be employed.

The duration of the heat treatment is at least 5 minutes, preferably more than 10 minutes, more preferably at least 0.25 hours, in particular at least 0.5 hours, and more in particular at least 0.75 hours. Preferably, the duration of the heat treatment may be at most 100 hours, more preferably at most 75 hours, most preferably at most 60 hours, in particular in the range of from 0.25 to 50 hours, more in particular from 0.75 to 40 hours.

The feed, hereinafter “treatment feed” which may be employed in the heat treatment may be any oxygen containing feed. Preferably, the treatment feed may be pure oxygen or it may comprise additional components which are inert under the prevailing conditions. Suitably, the treatment feed may be a mixture comprising oxygen and an inert gas, such as argon, carbon dioxide, helium, nitrogen, or a saturated hydrocarbon. Such mixtures may be, for example, air, oxygen enriched air, or air/methane mixtures. Suitably, in addition to oxygen, the treatment feed may comprise one or more olefins, such olefins are described hereinafter. Such mixtures may be dehumidified or humidified, preferably humidified. However, the presence of one or more of these additional components in the treatment feed is not considered to be essential to the invention.

The quantity of oxygen in the treatment feed may preferably be in the range of from 1 to 30% v, more preferably from 2 to 25% v, most preferably from more than 3 to 25% v, relative to the total feed. The quantity of inert gas may be in the range of from 99 to 70% v, in particular from 98 to 75% v, more in particular from less than 97 to 75% v, relative to the total treatment feed.

The heat treatment may typically be carried at an absolute pressure of up to 4000 kPa, preferably in the range of from 50 to 2000 kPa, for example 101.3 kPa (atmospheric pressure).

The present heat treatment may preferably be conducted as a separate process, in other words not incorporated as a step in an epoxidation process, due to the temperature constraints of typical commercial plants.

The heat treatment of the catalyst may be carried out by a method wherein the catalyst, or a precursor of the catalyst, is supplied to a heating apparatus and contacted with the heated treatment feed gas. The heat treatment may be a batch-type process or a continuous process. The heating apparatus may be an oven, a kiln or the like, or preferably, a gas flow band dryer. With a gas flow band dryer, the catalyst to be heat treated is put on a gas flow type endless belt and transported in the dryer while the heated treatment feed gas is passed through the object to be dried from an upper or lower direction of the belt. For further reference see “Perry's Chemical Engineers' Handbook” by Robert H. Perry et al. 6^{th} ed. pages 20-14 to 20-51 (1984).

The treatment feed gas may be recycled to increase process efficiency. The treatment feed, after contact with the catalyst, or a precursor of the catalyst, in the heating apparatus, may be withdrawn and introduced again. Before reintroduction into the heating apparatus, a part of the withdrawn gas may be purged and replaced with fresh treatment feed gas to avoid accumulation of contaminants in the treatment feed.

Subsequent to the heat treatment, the catalyst temperature may be decreased to a catalyst temperature of at most 325° C., preferably at most 310° C., more preferably below 300° C. The gaseous content may be maintained the same as the treatment feed, replaced by an epoxidation feed, as described hereinafter, or replaced with an inert gas, as described hereinbefore. The pressure may also be maintained the same as for the heat treatment, increased or decreased.

Preferably, the catalyst temperature may be decreased to a temperature which may be suitable for storage of the catalyst, for example a catalyst temperature in the range of from 0 and 50° C., in particular from 10 to 40° C. Preferably, the catalyst may be stored in the presence of an inert gas. After storage, the catalyst may be applied in an epoxidation process.

In an embodiment, the heat treatment may be carried out as part of the epoxidation process involving a packed catalyst bed, so long as it is possible for the epoxidation equipment to reach the required catalyst temperature. The GHSV of the heated treatment feed may be in the range of from 1500 to 10000 Nl/(l.h). “GHSV” or Gas Hourly Space Velocity is the unit volume of gas at normal temperature and pressure (0° C., 1 atm, i.e. 101.3 kPa) passing over one unit volume of packed catalyst per hour. The heat treatment may be incorporated in the epoxidation process in any phase of the epoxidation process, for example during the start up or during the regular olefin oxide production. Following the heat treatment of the packed catalyst bed, the catalyst temperature may be decreased to a catalyst temperature of at most 325° C., preferably at most 310° C., more preferably below 300° C.

The following description relates to an epoxidation process which employs a catalyst having been subjected to the heat treatment of the invention. The epoxidation process may be carried out by using methods known in the art. Reference may be made, for example, to U.S. Pat. No. 4,761,394, U.S. Pat. No. 4,766,105, U.S. Pat. No. 6,372,925 B1, U.S. Pat. No. 4,874,879 and U.S. Pat. No. 5,155,242, which are incorporated herein by reference.

Although the epoxidation process may be carried out in many ways, it is preferred to carry it out as a gas phase process, i.e. a process in which the epoxidation feed is contacted in the gas phase with the shaped catalyst which is present as a solid material, typically in a packed bed. Generally the process is carried out as a continuous process.

The olefin for use in the present epoxidation process may be any olefin, such as an aromatic olefin, for example styrene, or a di-olefin, whether conjugated or not, for example 1,9-decadiene or 1,3-butadiene. Mixtures of olefins may be used. Typically, the olefin may be a monoolefin, for example 2-butene or isobutene. Preferably, the olefin may be a mono-α-olefin, for example 1-butene or propylene. The most preferred olefin is ethylene.

The olefin concentration in the epoxidation feed may be selected within a wide range. Typically, the olefin concentration in the epoxidation feed will be at most 80 mole %, relative to the total feed. Preferably, it will be in the range of from 0.5 to 70 mole %, in particular from 1 to 60 mole %, on the same basis. As used herein, the epoxidation feed is considered to be the composition which is contacted with the catalyst.

The epoxidation process may be air-based or oxygen-based, see “Kirk-Othmer Encyclopedia of Chemical Technology”, 3^{rd} edition, Volume 9, 1980, pp. 445-447. In the air-based process air or air enriched with oxygen is employed as the source of the oxidizing agent while in the oxygen-based processes high-purity (at least 95 mole %) oxygen is employed as the source of the oxidizing agent. Presently most epoxidation plants are oxygen-based and this is a preferred embodiment of the present invention.

The oxygen concentration in the epoxidation feed may be selected within a wide range. However, in practice, oxygen is generally applied at a concentration which avoids the flammable regime. Typically, the concentration of oxygen applied will be within the range of from 1 to 15 mole %, more typically from 2 to 12 mole % of the total epoxidation feed.

In order to remain outside the flammable regime, the concentration of oxygen in the epoxidation feed may be lowered as the concentration of the olefin is increased. The actual safe operating ranges depend, along with the epoxidation feed composition, also on the reaction conditions such as the reaction temperature and the pressure.

A reaction modifier may be present in the epoxidation feed for increasing the selectively, suppressing the undesirable oxidation of olefin or olefin oxide to carbon dioxide and water, relative to the desired formation of olefin oxide. Many organic compounds, especially organic halides and organic nitrogen compounds, may be employed as the reaction modifier. Nitrogen oxides, hydrazine, hydroxylamine or ammonia may be employed as well. It is frequently considered that under the operating conditions of olefin epoxidation the nitrogen containing reaction modifiers are precursors of nitrates or nitrites, i.e. they are so-called nitrate- or nitrite-forming compounds (cf. e.g. EP-A-3642 and U.S. Pat. No. 4,822,900, which are incorporated herein by reference).

Organic halides are the preferred reaction modifiers, in particular organic bromides, and more in particular organic chlorides. Preferred organic halides are chlorohydrocarbons or bromohydrocarbons. More preferably they are selected from the group of methyl chloride, ethyl chloride, ethylene dichloride, ethylene dibromide, vinyl chloride or a mixture thereof. Most preferred reaction modifiers are ethyl chloride and ethylene dichloride.

Suitable nitrogen oxides are of the general formula NOₓ wherein x is in the range of from 1 to 2, and include for example NO, N₂O₃ and N₂O₄. Suitable organic nitrogen compounds are nitro compounds, nitroso compounds, amines, nitrates and nitrites, for example nitromethane, 1-nitropropane or 2-nitropropane. In preferred embodiments, nitrate- or nitrite-forming compounds, e.g. nitrogen oxides and/or organic nitrogen compounds, are used together with an organic halide, in particular an organic chloride.

The reaction modifiers are generally effective when used in low concentration in the epoxidation feed, for example up to 0.1 mole %, relative to the total feed, for example from 0.01×10⁻⁴ to 0.01 mole %. In particular when the olefin is ethylene, it is preferred that the reaction modifier is present in the epoxidation feed at a concentration of from 0.01×10⁻⁴ to 50×10⁻⁴ mole %, in particular from 0.3×10⁻⁴ to 30×10⁻⁴ mole %, relative to the total feed.

In addition to the olefin, oxygen and the reaction modifier, the epoxidation feed may contain one or more optional components, such as carbon dioxide, inert gases and saturated hydrocarbons. Carbon dioxide is a by-product in the epoxidation process. However, carbon dioxide generally has an adverse effect on the catalyst activity. Typically, a concentration of carbon dioxide in the epoxidation feed in excess of 25 mole %, preferably in excess of 10 mole %, relative to the total feed, is avoided. A concentration of carbon dioxide as low as 1 mole % or lower, relative to the total epoxidation feed, may be employed. Inert gases, for example nitrogen or argon, may be present in the epoxidation feed in a concentration of from 30 to 90 mole %, typically from 40 to 80 mole %. Suitable saturated hydrocarbons are methane and ethane. If saturated hydrocarbons are present, they may be present in a quantity of up to 80 mole %, relative to the total epoxidation feed, in particular up to 75 mole %. Frequently they are present in a quantity of at least 30 mole %, more frequently at least 40 mole %. Saturated hydrocarbons may be added to the epoxidation feed in order to increase the oxygen flammability limit.

The epoxidation process may be carried out using reaction temperatures selected from a wide range. Preferably the reaction temperature is in the range of from 150 to 325° C., more preferably in the range of from 180 to 300° C.

The epoxidation process is preferably carried out at a reactor inlet pressure in the range of from 1000 to 3500 kPa. Preferably, when the epoxidation process is as a gas phase process involving a packed bed of the shaped catalyst particles, the GHSV may be in the range of from 1200 to 12000 Nl/(l.h), and, more preferably, GSHV is in the range of from 1500 to less than 10000 Nl/(l.h). Preferably, the process is carried out at a work rate in the range of from 0.5 to 10 kmole olefin oxide produced per m³ of catalyst per hour, in particular 0.7 to 8 kmole olefin oxide produced per m³ of catalyst per hour. As used herein, the work rate is the amount of the olefin oxide produced per unit volume of the packed bed of the shaped catalyst particles per hour and the selectivity is the molar quantity of the olefin oxide formed relative to the molar quantity of the olefin converted.

The olefin oxide produced may be recovered from the reaction mixture by using methods known in the art, for example by absorbing the olefin oxide from a reactor outlet stream in water and optionally recovering the olefin oxide from the aqueous solution by distillation. At least a portion of the aqueous solution containing the olefin oxide may be applied in a subsequent process for converting the olefin oxide into a 1,2-diol or a 1,2-diol ether.

The olefin oxide produced in the epoxidation process may be converted into a 1,2-diol, a 1,2-diol ether, or an alkanolamine. As this invention leads to a more attractive process for the production of the olefin oxide, it concurrently leads to a more attractive process which comprises producing the olefin oxide in accordance with the invention and the subsequent use of the obtained olefin oxide in the manufacture of the 1,2-diol, 1,2-diol ether, and/or alkanolamine.

The conversion into the 1,2-diol or the 1,2-diol ether may comprise, for example, reacting the olefin oxide with water, suitably using an acidic or a basic catalyst. For example, for making predominantly the 1,2-diol and less 1,2-diol ether, the olefin oxide may be reacted with a ten fold molar excess of water, in a liquid phase reaction in presence of an acid catalyst, e.g. 0.5-1.0% w sulfuric acid, based on the total reaction mixture, at 50-70° C. at 1 bar absolute, or in a gas phase reaction at 130-240° C. and 20-40 bar absolute, preferably in the absence of a catalyst. If the proportion of water is lowered the proportion of 1,2-diol ethers in the reaction mixture is increased. The 1,2-diol ethers thus produced may be a di-ether, tri-ether, tetra-ether or a subsequent ether. Alternative 1,2-diol ethers may be prepared by converting the olefin oxide with an alcohol, in particular a primary alcohol, such as methanol or ethanol, by replacing at least a portion of the water by the alcohol.

The conversion into the alkanolamine may comprise, for example, reacting the olefin oxide with ammonia. Anhydrous or aqueous ammonia may be used, although anhydrous ammonia is typically used to favour the production of monoalkanolamine. For methods applicable in the conversion of the olefin oxide into the alkanolamine, reference may be made to, for example U.S. Pat. No. 4,845,296, which is incorporated herein by reference.

The 1,2-diol and the 1,2-diol ether may be used in a large variety of industrial applications, for example in the fields of food, beverages, tobacco, cosmetics, thermoplastic polymers, curable resin systems, detergents, heat transfer systems, etc. The alkanolamine may be used, for example, in the treating (“sweetening”) of natural gas.

Unless specified otherwise, the low-molecular weight organic compounds mentioned herein, for example the olefins, 1,2-diols, 1,2-diol ethers, alkanolamines and reaction modifiers, have typically at most 40 carbon atoms, more typically at most 20 carbon atoms, in particular at most 10 carbon atoms, more in particular at most 6 carbon atoms. As defined herein, ranges for numbers of carbon atoms (i.e. carbon number) include the numbers specified for the limits of the ranges.

Having generally described the invention, a further understanding may be obtained by reference to the following examples, which are provided for purposes of illustration only and are not intended to be limiting unless otherwise specified.

### EXAMPLES

Carrier A was prepared according to the method outlined in US 2003/0162984 A1 for “Carrier B”. The resulting carrier, Carrier A, exhibited the following characteristics:

| | | |
|---|---|---|
| Surface Area: | 2.16 | m²/g |
| Water Absorption: | 0.49 | g/g |
| Pore Volume: | 0.42 | ml/g |
| Pore Size Distribution: | | |
| <0.2 μm | 9% | v |
| 0.2–10 μm | 72% | v |
| >10 μm (% v) | 19% | v |

The pore size distribution is specified as the volume fraction (% v) and the volume (ml/g) of the pores having diameters in the range of from 0.2-10 μm is about 0.3 ml/g, relative to the total pore volume. “Pore volume” represents the total pore volume.

### Preparation of Catalyst

Catalyst A was prepared using Carrier A by a similar method as outlined in US 2003/0162984 A1 to yield a finished catalyst having 18% w silver, relative to the total weight of the catalyst; 7.5 mmoles cesium per kg of catalyst; 2 mmoles of rhenium per kg of catalyst; 1 mmole tungsten per kg of catalyst; and 40 mmoles lithium per kg of catalyst.

### Catalyst Heat Treatment

A portion of Catalyst A so prepared was then placed in a forced air oven and heated to 400° C. The catalyst was heated at 400° C. for 45 minutes in an air stream and then cooled to room temperature. The resulting catalyst was Catalyst B, according to the invention. The portion of the prepared catalyst not subjected to the heat treatment was Catalyst A, comparative.

### Catalyst Performance Testing

### Example 1

### According to the Invention

Catalyst B was used to produce ethylene oxide from ethylene and oxygen. To do this, 1.7 g of crushed catalyst were loaded into a stainless steel U-shaped tube (3.86 mm inner diameter). The tube was immersed in a molten metal bath (heat medium) and the ends were connected to a gas flow system. The weight of catalyst used and the inlet gas flow rate were adjusted to give a gas hourly space velocity of 6800 Nl/(l.h). The inlet gas pressure was 1550 kPa.

The gas mixture passed through the catalyst bed, in a “once-through” operation, during the entire test run and consisted of 30% v ethylene, 8% v oxygen, 5% v carbon dioxide, 57% v nitrogen. Ethyl chloride was also added to the gas mixture. The ethyl chloride was added to the epoxidation feed in a low quantity and was increased to a value of 1.7 ppmv ethyl chloride.

The initial reaction temperature was 180° C. and this was ramped up at a rate of 10° C. per hour to 225° C. and then adjusted so as to achieve a constant ethylene oxide content of 1.7% v in the outlet gas stream.

The initial selectivity was 87.6% which occurred at a corresponding reaction temperature of 254° C.

### Example 2

### Comparative

Catalyst A was used to produce ethylene oxide from ethylene and oxygen. To do this, 1.7 g of crushed catalyst were loaded into a stainless steel U-shaped tube (3.86 mm inner diameter). The tube was immersed in a molten metal bath (heat medium) and the ends were connected to a gas flow system. The catalyst in the reactor was maintained at 280° C. for 32 hours under a flow of air, i.e., treatment feed, at GHSV of 6800 Nl/(l.h). The catalyst temperature was decreased to 200° C., the air feed to the catalyst was replaced by a feed of 30% v ethylene, 8% v oxygen, 5% v carbon dioxide, 57% v nitrogen, and subsequently ethyl chloride was added to the epoxidation feed in a low quantity and was increased to a value of 1.7 ppmv. The inlet gas flow rate was maintained at a gas hourly space velocity of 6800 Nl/(l.h). The inlet gas pressure was 1550 kPa. The reaction temperature was then adjusted so as to achieve a constant ethylene oxide content of 1.7% v in the outlet gas stream. The gas mixtures passed through the catalyst bed, in a “once-through” operation, during the entire process.

The initial selectivity was 83.8% which occurred at a corresponding reaction temperature of 230° C.

### Example 3

### Comparative

Catalyst A was tested according to Example 2 except the catalyst in the reactor was maintained at 280° C. for 200 hours under a flow of air at GHSV of 6800 Nl/(l.h).

The initial selectivity was 84.1% which occurred at a corresponding reaction temperature of 233° C.

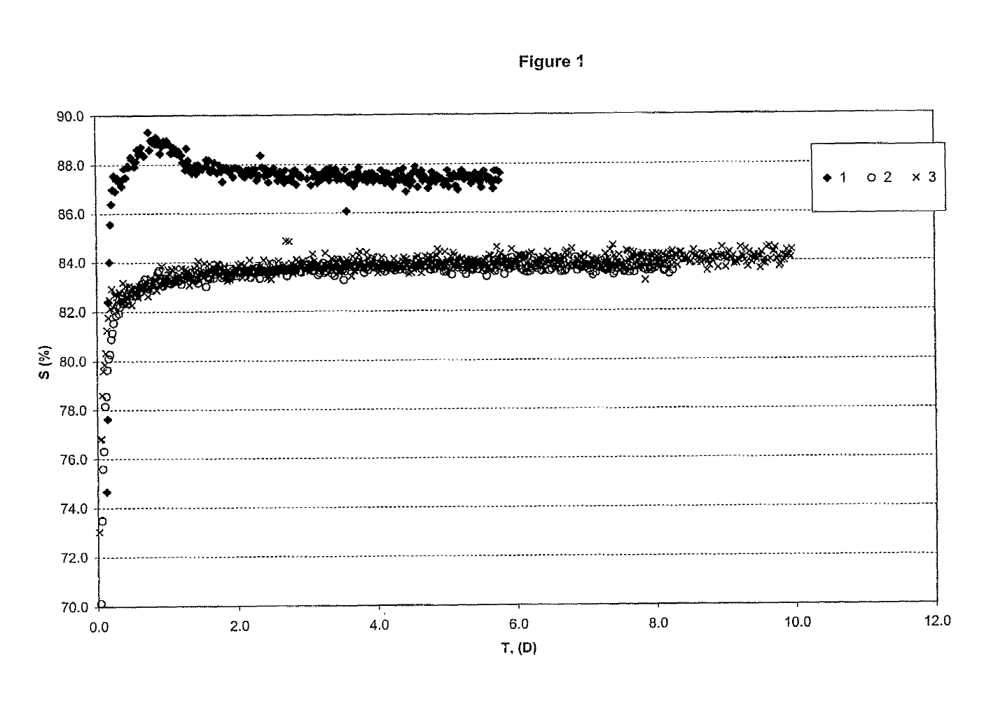

Reference is made to FIG. 1. FIG. 1 shows that in Example 1 the heat treatment according to the invention results in a catalyst which initially operates at a higher selectivity than a catalyst heat treated at a lower temperature as described in Examples 2 and 3.

### Catalyst Attrition Testing

### Example 4

Catalyst A and Catalyst B were tested in accordance with ASTM D4058-96 with the elimination of the drying step for the sample. Catalyst A (comparative) had an attrition loss of 22% and Catalyst B (according to the invention) had an attrition loss of 20%. This demonstrates that the heat treatment according to the invention improves the attrition of the catalyst.

### Example 5

Carrier C was prepared according to the method outlined in US 2003/0162984 A1 for “Carrier A”. The resulting carrier, Carrier C, exhibited the following characteristics:

| | | |
|---|---|---|
| Surface Area: | 2.04 | m²/g |
| Water Absorption: | 0.42 | g/g |
| Pore Volume: | 0.41 | ml/g |
| Pore Size Distribution: | | |
| <0.2 μm | 5% | v |
| 0.2–10 μm | 92% | v |
| >10 μm (% v) | 3% | v |

The pore size distribution is specified as the volume fraction (% v) and the volume (ml/g) of the pores having diameters in the range of from 0.2-10 μm is about 0.37 ml/g, relative to the total pore volume. “Pore volume” represents the total pore volume.

Carrier C was then impregnated in a similar manner as outlined in as in WO 2005/097318 A1 for “Catalyst A” using double impregnation to yield Catalyst C having 26% w silver, relative to the total weight of the catalyst; 8.5 mmoles cesium per kg of catalyst; 2.5 mmoles of rhenium per kg of catalyst; 0.8 mmole tungsten per kg of catalyst; and 40 mmoles lithium per kg of catalyst.

A portion of Catalyst C was placed in a forced air oven and heated to 400° C. The catalyst was heated at 400° C. for 45 minutes in an air stream and then cooled to room temperature. The resulting catalyst was Catalyst D, according to the invention.

Catalyst C and Catalyst D were tested in accordance with ASTM D4058-96 with the elimination of the drying step for the sample. Catalyst C (comparative) had an attrition loss of 21% and Catalyst D (according to the invention) had an attrition loss of 17%.

This example demonstrates that the heat treatment according to the invention improves the attrition of a catalyst.

What is claimed is:

1. A process for treating a supported epoxidation catalyst comprising silver in a quantity of at most 0.15 g per m² surface area of the support, which process comprises:
contacting the catalyst, or a precursor of the catalyst comprising silver in cationic form, with a treatment feed comprising oxygen at a catalyst temperature of at least 350° C. for a duration of at least 5 minutes.

2. The process as claimed in claim 1, wherein the process further comprises subsequently decreasing the catalyst temperature to at most 325° C.

3. The process as claimed in claim 1, wherein the catalyst comprises an α-alumina support having a surface area of at least 1 m²/g, and a pore size distribution such that pores with diameters in the range of from 0.2 to 10 μm represent at least 70% of the total pore volume and such pores together provide a pore volume of at least 0.25 ml/g, relative to the weight of the support.

4. The process as claimed in claim 1, wherein the catalyst comprises an α-alumina support having a surface area of at least 1 m²/g, and a pore size distribution such that the median pore diameter is more than 0.8 μm, and such that at least 80% of the total pore volume is contained in pores with diameters in the range of from 0.1 to 10 μm, and at least 80% of the pore volume contained in the pores with diameters in the range of from 0.1 to 10 μm is contained in pores with diameters in the range of from 0.3 to 10 μm.

5. The process as claimed in claim 1, wherein the catalyst comprises, in addition to silver, a Group IA metal, and one or more selectivity enhancing dopants.

6. The process as claimed in claim 1, wherein the catalyst comprises, in addition to silver, rhenium or compound thereof, and a further metal or compound thereof selected from the group consisting of Group IA metals, Group IIA metals, molybdenum, tungsten, chromium, titanium, hafnium, zirconium, vanadium, thallium, thorium, tantalum, niobium, gallium, germanium, and mixtures thereof.

7. The process as claimed in claim 6, wherein the catalyst further comprises a rhenium co-promoter selected from the group consisting of sulfur, phosphorus, boron, and compounds thereof.

8. The process as claimed in claim 1, wherein in the catalyst comprises an α-alumina support and the quantity of silver relative to the surface area of the support is at most 0.12 g/m².

9. The process as claimed in claim 1, wherein in the catalyst comprises silver in a quantity of from 10 to 500 g/kg, on the total catalyst, and the support has a surface area of from 1.5 to 5 m²/g.

10. The process as claimed in claim 1, wherein in the treatment feed comprises oxygen in a quantity of from 1 to 30% v, relative to the total feed, and the catalyst temperature is in the range of from 350° C. to 700° C.

11. The process as claimed in claim 1, wherein the catalyst, or a precursor of the catalyst comprising the silver in cationic form, is contacted at a catalyst temperature in the range of from 375° C. to 600° C. for a duration of 0.25 to 50 hours.

12. The process as claimed in claim 1, wherein the attrition loss of the treated catalyst is at most 30%.

13. The process as claimed in claim 1, wherein the attrition loss of the treated catalyst is at most 20%.

14. A catalyst obtainable by the process according to claim 1.

15. A process for the epoxidation of an olefin, which process comprises contacting an epoxidation feed comprising the olefin and oxygen with a catalyst according to claim 13.

16. The process as claimed in claim 14, wherein the olefin comprises ethylene.

17. The process as claimed in claim 14, wherein the epoxidation feed additionally comprises, as a reaction modifier, an organic chloride and optionally a nitrate- or nitrite-forming compound.

18. A process for producing a 1,2-diol, a 1,2-diol ether or an alkanolamine comprising converting the olefin oxide into the 1,2-diol, the 1,2-diol ether, or the alkanolamine, wherein the olefin oxide has been obtained by a process for the epoxidation of an olefin according to claim 15.
